(19) United States
(12) Patent Application Publication
(10) Pub. No.: US 2022/0215933 A1
(43) Pub. Date: Jul. 7, 2022

(54) REMOTE EMDR THERAPY DEVICE

(71) Applicant: Mom's Favorite, PBC, Alameda, CA (US)

(72) Inventors: Rudy Samuel Shrier, Denver, CO (US); Stephen James Frouge, Tampa, FL (US)

(21) Appl. No.: 17/569,667

(22) Filed: Jan. 6, 2022

Related U.S. Application Data
(60) Provisional application No. 63/134,540, filed on Jan. 6, 2021.

(51) Int. Cl. G16H 20/70; A61M 21/02

(52) U.S. Cl. CPC G16H 20/70; A61M 2021/0022; A61M 21/02

(57) ABSTRACT

Apparatuses, methods, and systems for providing remote EMDR therapy to a patient. A remote EMDR device comprises a first handheld assembly and a second handheld assembly, each handheld assembly comprising one or more visual and/or tactile stimulation components A microcontroller module in the remote EMDR device is operably connected to the first and second handheld assemblies and is configured to establish a connection to a computing device of the patient that can receive control instructions from a remote therapist over the Internet. The remote EMDR device may then administer bilateral stimulation to the patient through the one or more visual and tactile stimulation components of the first and second handheld assemblies in accordance with the received control instructions.

### CROSS-REFERENCE TO RELATED APPLICATIONS

This application claims the benefit of U.S. Provisional Application No. 63/134,540 filed Jan. 6, 2021, entitled “REMOTE EMDR THERAPY DEVICE,” the entire disclosure of which is hereby incorporated herein by this reference.

### BRIEF SUMMARY

The present disclosure relates to technologies for providing a remotely controlled Eye Movement Desensitization and Reprocessing (“EMDR”) therapy device. According to some embodiments, a remote EMDR device comprises a first handheld assembly and a second handheld assembly, each handheld assembly comprising one or more of a visual stimulation component and a tactile stimulation component. A microcontroller module is operably connected to the first and second handheld assemblies and configured to establish a connection to a computing device of the patient that can receive control instructions from a remote therapist over the Internet. The remote EMDR device may then administer bilateral stimulation to the patient through the one or more visual and tactile stimulation components of the first and second handheld assemblies in accordance with the received control instructions.

According to further embodiments, a method comprises connecting, by a computing device of a patient, to a cloud server hosting a therapist web application over one or more networks. The computing device further connects to a remote EMDR device in possession of the patient. The therapist web application receives control instructions for the remote EMDR device from a therapist and sends the control instructions to the computing device over the one or more networks. The computing device forwards the control instructions to the remote EMDR device, and the remote EMDR device administers bilateral stimulation to the patient through one or more visual and tactile stimulation components of the remote EMDR device based on the forwarded control instructions.

According to further embodiments, a remote EMDR therapy system comprises a remote EMDR device, a patient mobile device, and a cloud server. The cloud server is operably connected to the patient mobile device over one or more networks and hosts a therapist web application configured to receive control instructions for the remote EMDR device from a therapist via a therapist console, and send the control instructions to the patient mobile device over the one or more networks. The patient mobile device is operably connected to the remote EMDR device and is configured to forward the control instructions received from the cloud server to the remote EMDR device. The remote EMDR device comprises a microcontroller module, a first handheld assembly, and a second handheld assembly, each handheld assembly operably connected to the microcontroller module and comprising one or more visual and/or tactile stimulation components. The microcontroller module is configured to administer bilateral stimulation to the patient through the one or more visual and tactile stimulation component of the handheld assemblies based on the control instructions forwarded by the patient mobile device.

These and other features and aspects of the various embodiments will become apparent upon reading the following Detailed Description and reviewing the accompanying drawings.

### BRIEF DESCRIPTION OF THE DRAWINGS

In the following Detailed Description, references are made to the accompanying drawings that form a part hereof, and that show, by way of illustration, specific embodiments or examples. The drawings herein are not drawn to scale, and any measurements provided are shown to provide a relative size context and are not intended to be limiting. Like numerals represent like elements throughout the several figures.

FIG. 1 is a block diagram showing an exemplary remote EMDR device and an associated patient mobile device, according to embodiments presented herein.

FIG. 2 is a system diagram showing the components of an exemplary remote EMDR therapy system, according to embodiments presented herein.

FIGS. 3A and 3B are GUI diagrams showing exemplary user interfaces of a therapist web application as part of the remote EMDR therapy system, according to embodiments presented herein.

FIG. 4 is a block diagram showing an example system and software architecture for the remote EMDR device and other computing devices described herein.

FIG. 5 is a flow chart showing an exemplary routine for utilizing a remote EMDR therapy system to facilitate a remote EMDR therapy session, according to embodiments presented herein.

FIG. 6 is a flow chart showing an exemplary routine for processing instructions from a cloud server to remotely control a remote EMDR device, according to embodiments presented herein.

### DETAILED DESCRIPTION

The following detailed description is directed to technologies for providing a remote EMDR therapy device to patients. Since the 1980's, EMDR and EMDR research has required either both the therapist and client to be in the same room or for the client to make repetitive motions to stimulate their brain bilaterally as a primary piece of the therapeutic intervention, due to hardware limitations. As technology has improved, over time we have seen the addition of software which will mimic this by lighting up the left and right borders of a computer screen, causing the brain to stimulate bilaterally, and allowing this work to happen remotely for telehealth. However, many users struggle with eye movements from the inception, hence many therapists continue to utilize audio and tactile stimulation in conjunction with visual cues in many EMDR therapeutic sessions.

Utilizing the technologies described herein, a remote EMDR device, also referred to herein as the “EMDR Hero,” may be implemented to empower therapists to engage in EMDR protocol style therapy with a patient with bilateral stimulation both in tactile and visual cues without requiring the therapist and patient to be in the same room. To allow EMDR and EMDR research to move forward during times of reduced availability of in-person therapy sessions, a device is provided that connects via, e.g., Bluetooth and an app, to the patient's mobile device, such as a mobile phone or tablet. By doing so, it allows a therapist anywhere with an Internet connection to supply bilateral stimulation from tactile (e.g., vibration) and visual (e.g., LED) cues.

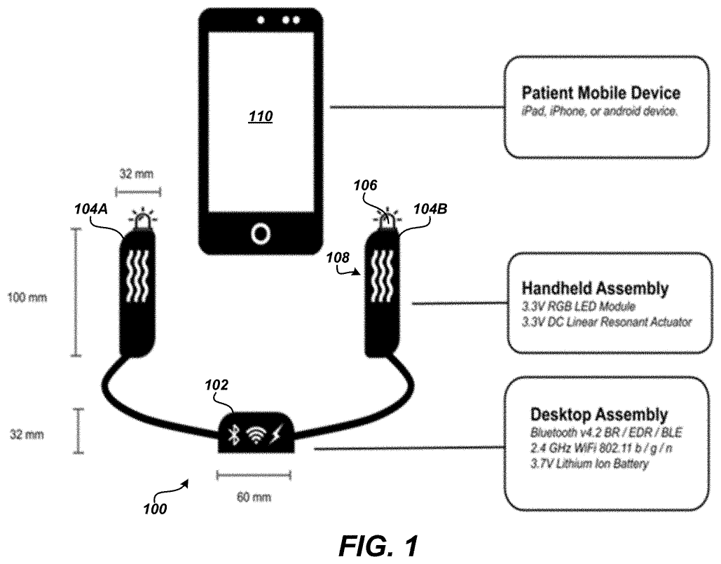

FIG. 1 show an exemplary remote EMDR device 100 and an associated patient mobile device 110, according to some embodiments. The remote EMDR device 100 comprises a desktop assembly 102 connected to two egg-shaped or bicycle grip-like handheld assemblies 104A, 104B (also referred to herein individually as left handheld assembly 104A and right handheld assembly 104B and collectively as handheld assemblies 104). According to embodiments, each handheld assembly 104A, 104B may include a visual stimulation component 106 for visual stimulation. In some embodiments, the visual stimulation component 106 may comprise one or more RGB LED modules, such as a series of WS2812 LED “pixels,” allowing full control of the color and intensity of the visual stimulation. Each handheld assembly 104A, 104B may further include a tactile stimulation component 108 for tactile stimulation, such as an integrated motor with counterweight, that when powered up will vibrate within the handheld assembly.

According to some embodiments, the handheld assemblies 104 may be identical to each other, or the left handheld assembly 104A and right handheld assembly 104B may be mirrors of each other, using the same form factor and components but shaped and/or configured for left-hand or right-hand use, respectively. The handheld assemblies 104 may be connected to the desktop assembly 102 through modular connectors and by varying lengths of wire, allowing the remote EMDR device 100 to be easily assembled by the end user (patient) and the handheld assemblies to be comfortably held at an effective distance apart by most any sized human. The modular connections also allow for easy modification of the remote EMDR device 100 to support the therapy for patients with a variety of physical disabilities. For example, the connection of each handheld assembly 104A, 104B to the desktop assembly 102 using telephone cable and standard RJ11 connectors.

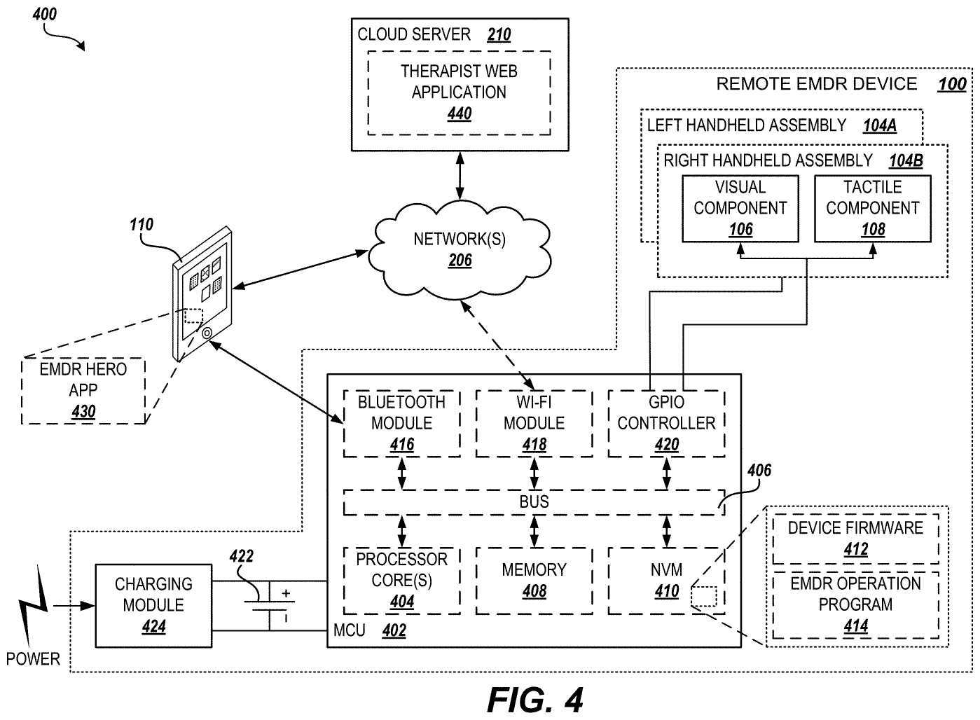

The desktop assembly 102 contains the electrical circuitry and components supporting remote control of the visual stimulation components 106 and tactile stimulation components 108 of the handheld assemblies 104. For example, in some embodiments the desktop assembly 102 may house a microcontroller and supporting circuitry allowing the remote EMDR device 100 to connect to the patient mobile device 110, such as a mobile phone or tablet device, allowing the mobile device and/or a remote application under the control of a therapist to control the operation of the EMDR device. The desktop assembly 102 may further include a battery and charging components for powering the remote EMDR device 100, as will be described below in more detail in regard to FIG. 4.

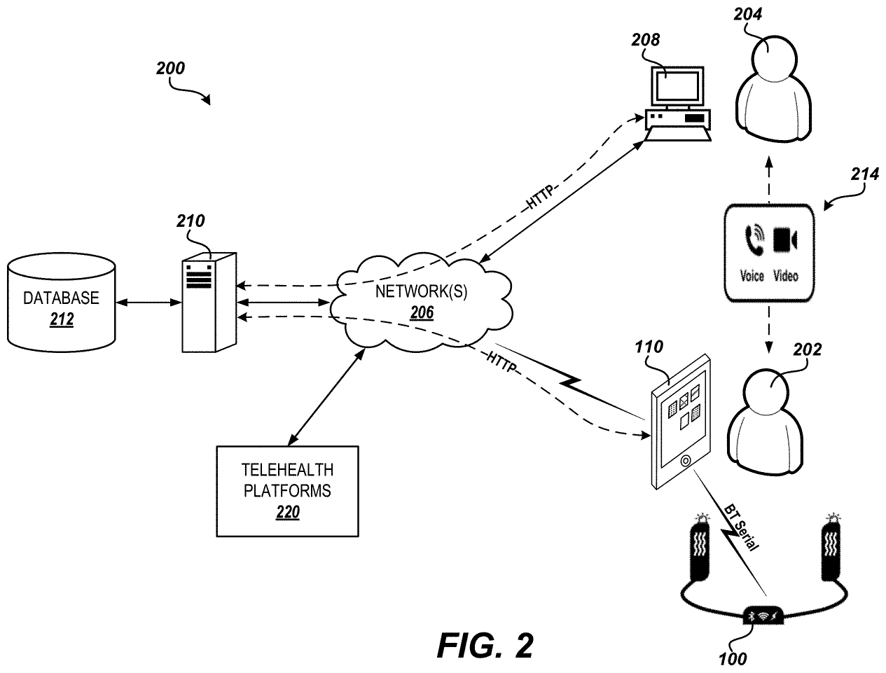

FIG. 2 shows an overview of a system 200 for providing remote EMDR therapy, according to some embodiments. According to embodiments, the system 200 includes a patient mobile device 110 of an end-user or “patient” 202 connected to a remote EMDR device 100. In some embodiments, the patient mobile device 110 may represent a mobile phone or tablet device, such as an IPHONE® or IPAD® from Apple Inc. or an Android™ phone from Google LLC. In other embodiments, the patient mobile device 110 may represent a laptop computer, desktop computer, or any other computing device of the patient 202 capable of connecting to the remote EMDR device 100 and the network(s) 206. The remote EMDR device 100 may be connected with the patient mobile device 110 using a wired or wireless connection. For example, in some embodiments the remote EMDR device 100 may be paired with the patient mobile device 110 using BLUETOOTH® technology. In other embodiments, the remote EMDR device 100 may be connected to the patient mobile device 110 using Wi-Fi® or by a cable.

The system 200 further includes a cloud server 210. In some embodiments, the cloud server 210 may represent virtualized computing resources available in the cloud, such as Amazon EC2 Cloud Computing services. In further embodiments, the cloud server 210 may represent one or more of conventional web server, application server, and/or other application hosting environments executing on one more server computers. According to embodiments, the cloud server 210 may host a web application, also referred to herein as the “therapist web application.” A therapist 204 may utilize a web browsing device 208, such as a tablet, laptop, or desktop computer executing a web browser application, to access the therapist web application executing on the cloud server 210. The therapist 204 may then utilize the therapist web application to connect to and remotely control the remote EMDR device 100 of a patient 202 over one or more networks 206 via the cloud server 210 and the patient mobile device 110. For example, the therapist web application may send instructions and/or request information to/from the patient mobile device 110 over the network(s) 206 utilizing the HTTP protocol, and the patient mobile device may forward the instructions/requests to the remote EMDR device 100 utilizing Bluetooth Characteristics and/or a serial interface over the Bluetooth connection. In further embodiments, the instructions/requests will be transmitted utilizing secure and encrypted protocols to protect any patient information.

The cloud server 210 may be further connected to a database 212 or other storage resources facilitating storage of patient records, remote EMDR therapy session logs, authorized remote EMDR devices for the therapist 204, and the like. In further embodiments, the cloud server 210 may have access over the network(s) 206 to third party telehealth platform 220 to facilitate the exchange of patient information, therapy information, billing information, and the like, as authorized by the patient 202.

According to embodiments, the patient 202 may utilize a mobile application or “app,” also referred to herein as the “EMDR Hero app,” on the patient mobile device 110 to connect to the remote EMDR device 100 and initiate a remote EMDR therapy session. In some embodiments, the patient 202 may download the app for free from the “app store” supporting the patient mobile device 110. The app may further connect to the therapist web application on the cloud server 210 over the network(s) 206. The patient 202 may then identify a particular therapist 204 for the administration of remote EMDR therapy, such as by inputting a unique ID of the therapist in the app, authorizing the therapist 204 to access the patient's individual remote EMDR device 100 connected to their patient mobile device 110.

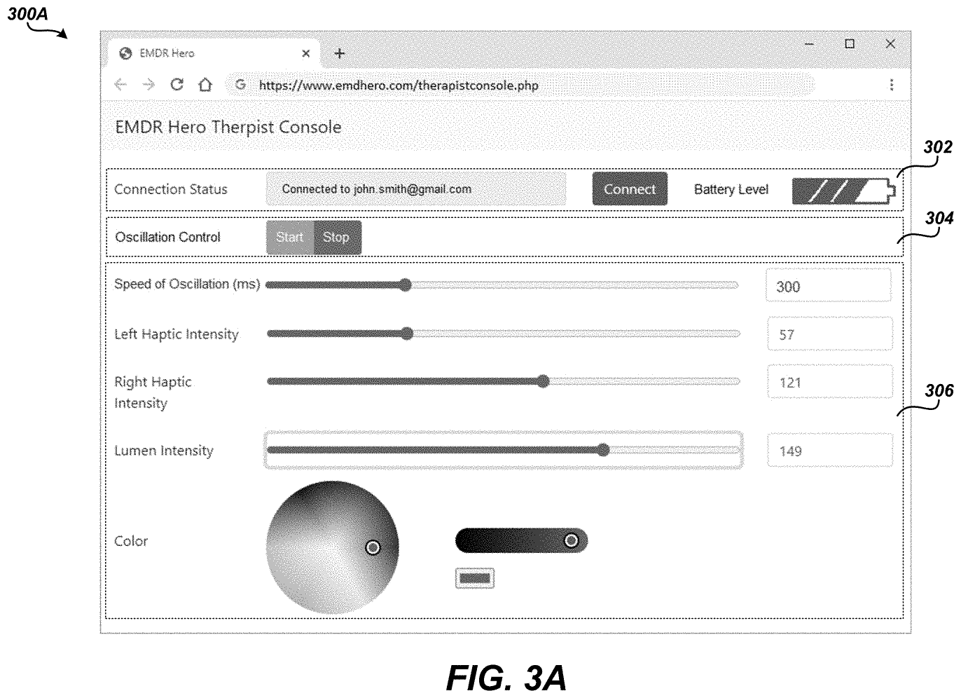

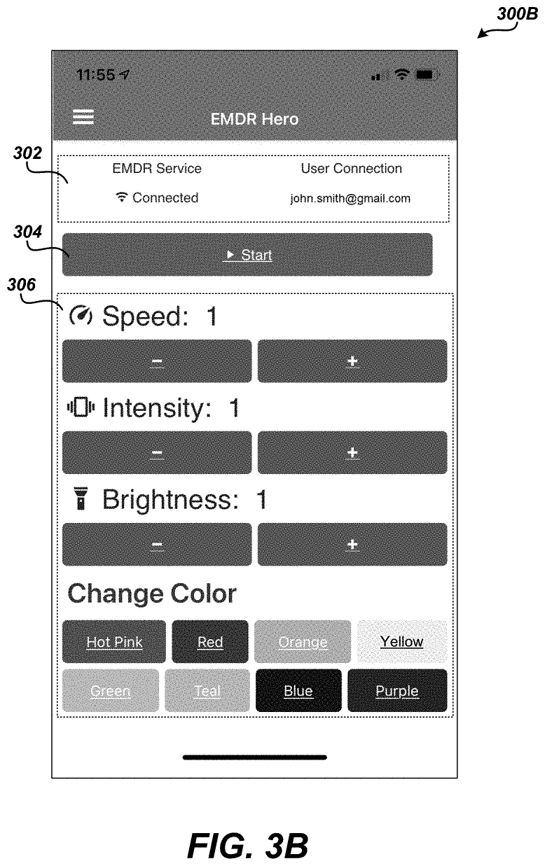

The therapist 204 may log into the therapist web application with a username and password and select the patient 202 from a list of all patients that have authorized access to their respected remote EMDR devices 100. Upon selection of the patient 202, the therapist may see a user interface showing a status of the patient's remote EMDR device 100 and allowing the therapist to remotely control the remote EMDR device 100 to facilitate the remote EMDR therapy session. For example, FIGS. 3A and 3B show example user interfaces 300A and 300B, alternatively referred to herein as “therapist console 300,” of the therapist web application allowing the therapist 204 to see status information 302, such as connection status and battery charge level, of the patient's remote EMDR device 100. The user interfaces 300A, 300B also contain user-interface control elements for controlling the remote EMDR device 100, such as a control to start and stop EMDR bilateral stimulation, as shown at 304, and controls to adjust a speed of oscillation of stimulation between the left and right handheld assemblies 104A and 104B, the intensity levels of the visual stimulation components 106 and tactile stimulation components 108 of the handheld assemblies, a color of the visual stimulation components 106, and the like, as shown at 306.

To facilitate a remote EMDR therapy session, the therapist 204 and patient 202 establish a voice and/or video call between them, as shown in FIG. 2 at 214. The voice/video call 214 may be facilitated by the therapist web application on the cloud server 210 and the EMDR Hero app on the patient mobile device 110, or the call may utilize more conventional web and/or phone based methods of voice/video calls. During the remote EMDR therapy session, the therapist 204 will remotely control the EMDR device so that one handheld assembly 104 at a time will light up and vibrate before oscillating to the other handheld assembly.

According to some embodiments, when the remote EMDR device 100 is powered on, the device will attempt to link with the patient mobile device 110 with which it is paired. The remote EMDR device 100 will then attempt to confirm that the patient mobile device 110 is connected to the cloud server 210 over the network(s) 206. The remote EMDR device 100 will then listen for device instructions from the therapist web application via the cloud server 210 and the patient mobile device 110. Instructions received by the remote EMDR device 100 may include terminating the session, changing the visual stimulation components 106 color, changing the intensity levels of the visual and/or tactile stimulation components 106 and 108, activating the visual bilateral stimulation, activating the tactile bilateral stimulation, and the like. In some embodiments, the remote EMDR device 100 will activate the visual stimulation component 106 and/or tactile stimulation component 108 of one handheld assembly 104A for between 300 milliseconds and 3,000 milliseconds before turning off and activating the other handheld assembly 104B. Before listening for additional instructions from the therapist web application, the remote EMDR device 100 may update the therapist web application on the cloud server 210 with the battery charge level, LED and vibration motor status, and the like.

FIG. 4 shows an example computing architecture 400 for implementation of the remote EMDR device 100 in the context of the remote EMDR therapy system 200, according to embodiments described herein. In some embodiments, the hardware architecture of the remote EMDR device 100 may utilize an integrated microcontroller module (also referred to herein as “MCU 402”), such as the ESP32-WROOM Wi-Fi/Bluetooth microcontroller module from Espressif Systems (Shanghai) Co., Ltd. The microcontroller module may be housed in the desktop assembly 102 of the remote EMDR device 100, for example.

The MCU 402 includes one or more processing core(s) 404. The processor core(s) 404 may represent standard programmable microprocessors that perform arithmetic and logical operations necessary for the operation of the remote EMDR device 100. The MCU 402 may further contain a data/control bus 406 that connects the processor core(s) 404 to the other integrated components of the microcontroller module. According to embodiments, the MCU 402 further includes a volatile memory 408. The memory 408 may include a random access memory (“RAM”) used as the main memory in the MCU 402, for example. The MCU 402 also includes a non-volatile memory, or NVM 410, such as flash memory, a non-volatile RAM (“NVRAM”), a read-only memory (“ROM”), or some combination of these and other non-volatile memory components. The NVM 410 may store processor-executable instructions and data in a non-transitory fashion for the operation of the MCU 402 in accordance with the embodiments described herein.

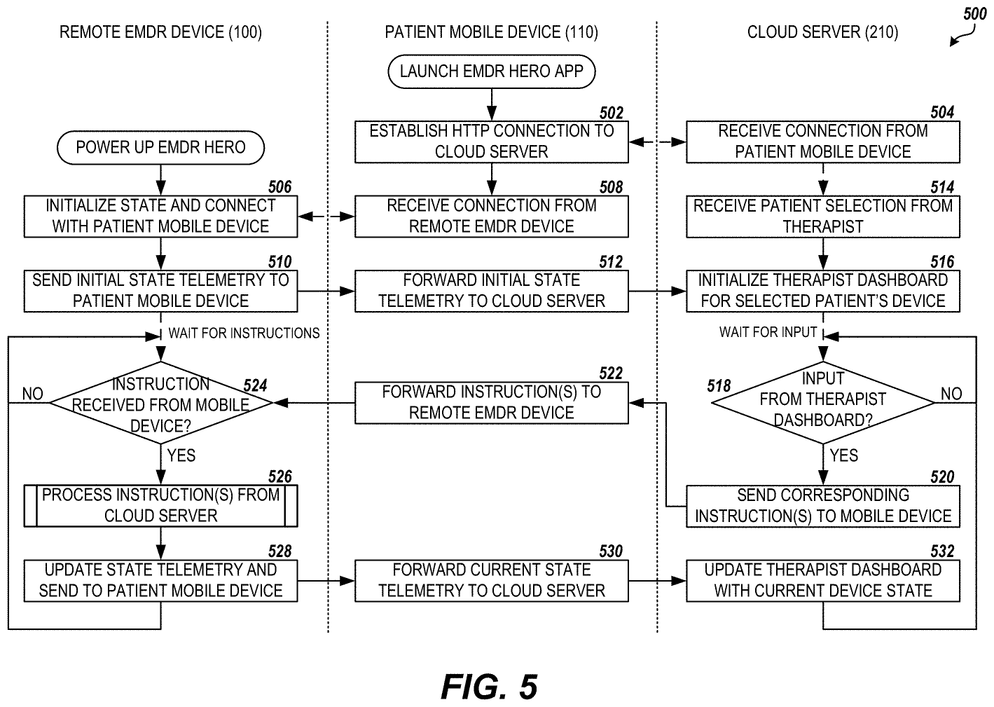

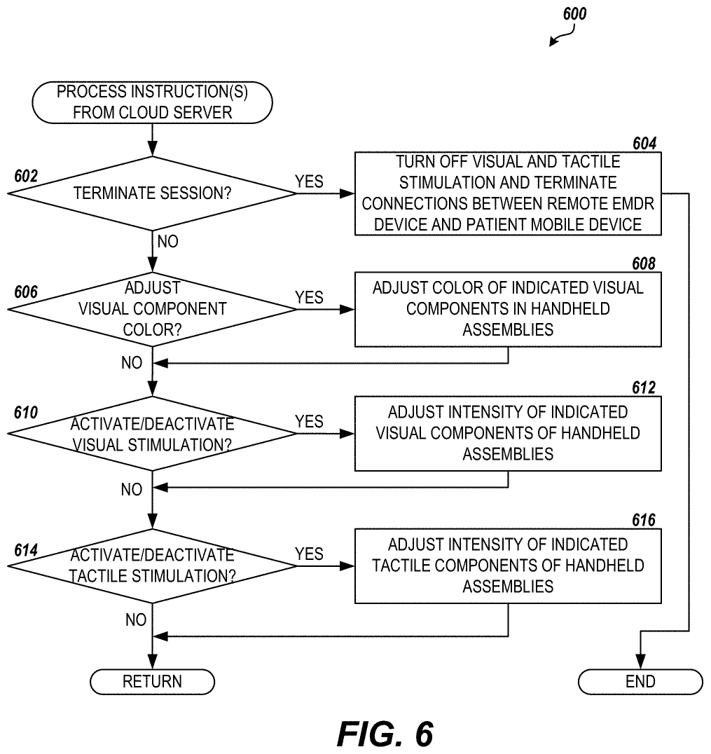

For example, the NVM 410 may store device firmware 412 containing basic routines that that help to startup the MCU 402 and transfer information between the various components and devices. The NVM 410 may also store other software components necessary for the operation of the remote EMDR device 100, according to embodiments. For example, the NVM 410 may store an EMDR operational program 414, also referred to as a “sketch,” that provides the functionality of the remote EMDR device 100 as part of the operation of the remote EMDR therapy system 200, as described herein. In other embodiments, the NVM 410 may be encoded with processor-executable instructions that, when executed by the MCU 402, perform the operations ascribed to the remote EMDR device 100 in the routines 500 and 600 described below in regard to FIGS. 5 and 6.

In some embodiments, the MCU 402 may further contain a Bluetooth module 416 that provides for Bluetooth pairing and communication between the remote EMDR device 100 and the patient mobile device 110 as described herein. In further embodiments, the MCU 402 may alternatively or additionally contain a Wi-Fi module 418, that facilitates connection over the network(s) 206 to the patient mobile device 110 and/or directly to the cloud server 210. In further embodiments, the MCU 402 may contain alternative or additional communication modules that facilitate connection of the remote EMDR device 100 to the patient mobile device 110 and/or cloud server either directly or through the network(s) 206, such as an Ethernet module, an LTE cellular module, a Universal Serial Bus (“USB”) interface module, a Serial Peripheral Interface (“SPI”) module, and the like. It will be appreciated that the network(s) 206 shown and described herein represent any networked environment that connect the remote EMDR device 100, the patient mobile device 110, the cloud server 210, and other remote computing devices and resources through one or more networks, such as a Wi-Fi network, a LAN, a WAN, a cellular data network, the Internet or “cloud,” or any other networking topology known in the art that connects computing devices together. as a gigabit Ethernet adapter, a Wi-Fi adapter, or an LTE data adapter.

According to further embodiments, the MCU 402 includes a GPIO controller 420 that facilitates connection of the microcontroller module to the handheld assemblies 104. For example, the MCU 402 may utilize pulse-width modulation over an assigned GPIO output to control operation and intensity of the tactile stimulation component 108, e.g., the vibration motors, in each of the left handheld assembly 104A and right handheld assembly 104B. Similarly, the MCU 402 may communicate with the visual stimulation components 106 of the handheld assemblies 104 utilizing a digital data output of the GPIO to control the color and intensity of the visual stimulation. It will be appreciated that the remote EMDR device 100 may contain additional circuitry and components, such as capacitors, resistors, transistors, and the like, that facilitate electrical interfacing of the GPIO controller 420 of the MCU 402 to the visual stimulation components 106 and tactical stimulation components 108 of the handheld assemblies 104.

In further embodiments, the remote EMDR device 100 may include a battery 422 housed in the desktop assembly 102. For example, the battery 422 may be a 3.7V Li-Ion battery module. In addition, the desktop assembly 102 may further house a battery charging module 424 to charge the battery 422 from an external power source. In some embodiments, the MCU 402 may utilize the GPIO controller 420 to read a charge level from the battery charging module 424 or directly from the battery 422 for reporting the current charge level to the therapist web application. In other embodiments the current charge level of the battery 422 may be determined from a voltage supplied to the MCU 402 by the battery 422.

Similarly to the NVM 410 of the MCU 402, the patient mobile device 110 and the cloud server 210 may be connected to computer-readable storage media storing application programs and data utilized in the remote EMDR therapy system 200. For example, the patient mobile device 110 may have access to computer-readable storage media storing the EMDR Hero app 430 utilized by the patient 202 to connect the remote EMDR device 100 to the cloud server 210 and initiate a remote EMDR therapy session, as described herein. The cloud server 210 may further have access to computer-readable storage media storing the therapist web application 440 utilized by the therapist 204 to remotely control the remote EMDR device 100. Examples of computer-readable storage media include, but are not limited to, hard disk drives, solid-state (Flash) drives, optical disk drives, magneto-optical disc drives, magnetic tape drives, memory cards, holographic memory, or any other computer-readable media known in the art that provides non-transitory storage of digital data and software. According to further embodiments, the computer-readable storage media may be encoded with processor-executable instructions that, when executed by processing resources of the patient mobile device 110 or cloud server 210, perform the operations ascribed to the patient mobile device and cloud server in the routines 500 and 600 described below in regard to FIGS. 5 and 6.

It will be appreciated that the computing architecture 400 may not include all of the components shown in FIG. 4, may include other components that are not explicitly shown in FIG. 4, or may utilize an architecture completely different than that shown in FIG. 4. For example, any of the processor core(s) 404, memory 408, NVM 410, Bluetooth module 416, Wi-Fi module 418, and GPIO controller 420 may represent separate components electrically connected together on one or more circuit boards or daughter boards. It is intended that all such computing architectures for the remote EMDR device 100 be included within the scope of this application.

FIG. 5 illustrates one routine 500 for utilizing the remote EMDR therapy system 200 to facilitate a remote EMDR therapy session, according to some embodiments. In some embodiments, parts of the routine 500 may be performed by the remote EMDR device 100, while other parts may be performed by the EMDR Hero app 430 executing on the patient mobile device 110 and by the therapist web application 440 executing on the cloud server 210, as shown in the figure. In other embodiments, the routine 500 may be performed by some combination of the remote EMDR device 100, the patient mobile device 110, the cloud server 210, and/or other computing devices, components, and modules of the remote EMDR therapy system 200. According to some embodiments, the routine 500 may be initiated by a patient 202 activating the EMDR Hero app 430 on the on their patient mobile device 110 to establish a remote EMDR therapy session, for example.

The routine 500 begins at step 502, where the EMDR Hero app 430 is launched by the patient 202 on the patient mobile device 110 and a connection with the cloud server 210 over the network(s) 206 is established. For example, the EMDR Hero app 430 may request a connection to the therapist web application 440 executing on the cloud server 210 over the Internet utilizing a secure HTTPS connection. The therapist web application 440 receives the request and establishes the connection, as shown at 504. From step 504, the routine 500 proceeds to steps 506, where the remote EMDR device 100 is powered on and establishes a connection with the patient mobile device 110. According to some embodiments, when the remote EMDR device 100 is powered on, it initializes the state of the device and then attempts to establish a Bluetooth connection with the patient mobile device 110 with which it is paired. The remote EMDR device 100 may then wait for confirmation that the patient mobile device 110 is connected and that the EMDR Hero app 430 is running on the mobile device and is connected to the cloud server 210.

As shown at step 510, once the remote EMDR device 100 is connected to the patient mobile device 110, the EMDR device sends its initial state telemetry to the mobile device. The initial state telemetry may include, for example, the current charge level of the battery 422. The initial state telemetry from the remote EMDR device 100 is then forwarded by the EMDR Hero app 430 to the cloud server 210, as further shown at 512. According to some embodiments, the initial state telemetry may be sent from the remote EMDR device 100 to the EMDR Hero app 430 using the Bluetooth connection, with the app forwarding the initial state telemetry to the cloud server 210 over the network(s) 206 using the HTTPS protocol. Once the remote EMDR device 100 has sent the initial state telemetry to the cloud server 210 via the patient mobile device 110, the device waits for instructions to be received from the cloud server 210 for operation. According to some embodiments, the remote EMDR device 100 may continue to periodically send updated state telemetry data to the cloud server 210 via the EMDR Hero app 430.

Meanwhile, as shown at step 514, a therapist 204 may have made a selection of the patient 202 for the remote EMDR therapy selection in the therapist web application 440 executing on the cloud server 210. From step 514, the routine 500 proceeds to step 516, where the therapist console 300 is initialized for the remote EMDR device 100 of the selected patient 202. Once the initial state telemetry data has been received from the patient mobile device 110 for the remote EMDR device 100, the therapist console 300 may be updated with the initial state of the device, such as the connection status and current charge level of the battery. The therapist web application 440 then waits for input from the therapist console 300 by the therapist 204. For example, the therapist 204 may issue remote control instructions to the remote EMDR device 100, such as toggling on or off the bilateral stimulation function, or adjusting the oscillation speed, the tactile stimulation intensity, the visual stimulation intensity, the color of the visual stimulation, or the like.

At step 518, if no input is received from the therapist console 300, the therapist web application 440 continues to wait for input from the therapist 204. If input is received from the therapist console 300, then the routine 500 proceeds to step 520, where the therapist web application 440 formats and sends one or more instructions to the patient mobile device 110 for the remote EMDR device 100 based on the input received from the therapist 204. The instructions may be sent to the patient mobile device 110 utilizing the HTTPS protocol. The EMDR Hero app 430 executing on the patient mobile device 110 receives the instruction(s) and forwards the instruction(s) to the remote EMDR device 100 over the Bluetooth connection, as further shown at step 522.

As shown at step 524, if an instruction is received by the remote EMDR device from the cloud server 210 via the patient mobile device 110, the routine 500 proceeds to step 526, where the remote EMDR device 100 processes the instruction. An example routine for processing remote instructions from the cloud server 210 is described below in regard to FIG. 6. According to some embodiments, after the instruction(s) from the cloud server 210 are processed in step 526, the routine 500 may proceed to step 528 where the remote EMDR device 100 updates its state telemetry data with, e.g., the current charge level of the battery 422, and send the current state telemetry data to the cloud server 210 via the patient mobile device 110, as shown at step 530. When the cloud server 210 receives update state telemetry data from the remote EMDR device 100, it may update the therapist console 300 with the current device state, as shown at step 532, before returning to waiting for input from the therapist console and sending new instructions. Similarly, after sending the current state telemetry to the cloud server 210 via the patient mobile device 110, the remote EMDR device 100 may return to waiting for further instructions from the cloud server.

FIG. 6 illustrates one routine 600 for processing instructions from the cloud server 210 to remotely control the remote EMDR device 100, as described above in regard to step 526. According to some embodiments, the routine 600 may be performed by the EMDR operation program 414 executing on the remote EMDR device 100 upon receiving the forwarded instruction(s) from the patient mobile device 110. In other embodiments, processing of remote control instructions may be performed by some combination of the EMDR Hero app 430 executing on the patient mobile device 110 and the EMDR operation program 414 executing on the remote EMDR device 100. For example, the EMDR Hero app 430 may receive the commands from the cloud server 210 via the HTTPS connection and make adjustments to Bluetooth Characteristics defined by the remote EMDR device 100 for the Bluetooth connection corresponding to the internal state of the device and components. The routine 600 begins at step 602, where it is determined whether the instruction(s) received from the cloud server 210 include an instruction to terminate the remote EMDR therapy session. If a terminate session instruction was received, the routine 600 proceeds to step 604, where the remote EMDR device 100 turns off the visual stimulation components 106 and tactile stimulation components 108 of the handheld assemblies 104. The remote EMDR device 100 and/or EMDR Hero app 430 may further terminate the Bluetooth connection between the device and the patient mobile device 110. From step 604, the routine 600 ends.

If the received instruction(s) do not include a terminate session instruction, then the routine 600 proceeds to step 606, where it is determined whether the instruction(s) received from the cloud server 210 include an instruction to adjust the color of one or more visual stimulation components 106 of the handheld assemblies 104. According to some embodiments, the adjust color instruction may specify a new color for the visual stimulation components 106 of both handheld assemblies 104, just the visual stimulation component of the left handheld assembly 104A, or just the visual stimulation component of the right handheld assembly 104B. If an adjust the color instruction was received, the routine 600 proceeds to step 608, where the remote EMDR device 100 and/or EMDR Hero app 430 updates the color configuration of the indicated visual stimulation components. This may be accomplished for example by writing an RGB color value to a color setting for each visual stimulation component.

Next, the routine 600 proceeds to step 610, where it is determined whether the instruction(s) received from the cloud server 210 include an instruction to activate or deactivate the visual stimulation in one or more of the handheld assemblies 104. For example, instructions may be sent alternatingly to activate the visual stimulation in the left handheld assembly 104A and then the visual stimulation in the right handheld assembly 104B based on the specified oscillation speed in the therapist console 300 to affect the bilateral stimulation. If an activate/deactivate visual stimulation instruction was received, the routine 600 proceeds to step 612, where the remote EMDR device 100 and/or EMDR Hero app 430 turns on or off the visual stimulation component 106 of the indicated handheld assembly(s) 104. This may be accomplished for example by writing an intensity value (e.g., “0” for off and “149” for on) to an intensity (e.g., brightness) setting for each visual stimulation component 106. In some embodiments, the instruction to activate or deactivate the visual stimulation may include a value for the intensity of the visual stimulation, or the command may simply specify that the visual stimulation should be turned on or off, with the remote EMDR device 100 and/or EMDR Hero app 430 using a prior, preset intensity value for the visual stimulation component(s) 106.

Next, the routine 600 proceeds to step 614, where it is determined whether the instruction(s) received from the cloud server 210 include an instruction to activate or deactivate the tactile stimulation in one or more of the handheld assemblies 104. As described above for the visual stimulation, instructions may be sent alternatingly to activate the tactile stimulation in the left handheld assembly 104A and then the tactile stimulation in the right handheld assembly 104B based on the specified oscillation speed in the therapist console 300 to effect the bilateral stimulation. If an activate/deactivate tactile stimulation instruction was received, the routine 600 proceeds to step 616, where the remote EMDR device 100 and/or EMDR Hero app 430 turns on or off the tactile stimulation component 108 of the indicated handheld assembly(s) 104. This may be accomplished for example by writing an intensity value (e.g., “0” for off and “121” for on) to an intensity (e.g., voltage) setting for each tactile stimulation component 108. Just as above, the instruction to activate or deactivate the tactile stimulation may include a value for the intensity of the tactile stimulation, or the command may simply specify that the tactile stimulation should be turned on or off, with the remote EMDR device 100 and/or EMDR Hero app 430 using a prior, preset intensity value for the tactile stimulation component 108. From step 616, the routine 600 ends.

Based on the foregoing, it will be appreciated that technologies for providing a remote EMDR therapy device are presented herein. While embodiments are generally described herein in regard to specific hardware and software components, it will be appreciated that the embodiments described in this disclosure may be implemented using any hardware or software components known in the art that implement the same described features/elements and accomplish the same described functions. This may include other single-board computers with or without additional add-on daughter boards using open architectures, a custom-built proprietary hardware circuit, and/or any combination of these and other hardware circuits. It may also include open source application packages and modules available as standard packages in an OS/FW distribution, additional open source application packages and modules added to the standard distribution, custom software modules and services coded in any language, and/or any combination of these and other software application modules. The above-described embodiments are merely possible examples of implementations, set forth for a clear understanding of the principles of the present disclosure.

Many variations and modifications may be made to the above-described embodiments without departing substantially from the spirit and principles of the present disclosure. All such modifications and variations are intended to be included within the scope of the present disclosure, and all possible claims to individual aspects or combinations and sub-combinations of elements or steps are intended to be supported by the present disclosure. The logical steps, functions or operations described herein as part of a routine, method or process may be implemented (1) as a sequence of processor-implemented acts, software modules or portions of code running on a microcontroller, computing device, or other computer system and/or (2) as interconnected machine logic circuits or circuit modules within the microcontroller, computing device, or other computer system. The implementation is a matter of choice dependent on the performance and other requirements of the system. Alternate implementations are included in which steps, operations or functions may not be included or executed at all, may be executed out of order from that shown or discussed, including substantially concurrently or in reverse order, depending on the functionality involved, as would be understood by those reasonably skilled in the art of the present disclosure.

It will be further appreciated that conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more particular embodiments or that one or more particular embodiments necessarily include logic for deciding, with or without user input or prompting, whether these features, elements and/or steps are included or are to be performed in any particular embodiment.

What is claimed is:

1. An apparatus for providing remote eye movement desensitization and reprocessing (“EMDR”) therapy comprising:
a first handheld assembly and a second handheld assembly, each handheld assembly comprising one or more of a visual stimulation component and a tactile stimulation component; and
a microcontroller module operably connected to the first and second handheld assemblies and configured to:
establish a connection to a computing device of a patient, the computing device being further connected to one or more networks,
receive control instructions from a remote therapist over the one or more networks via the computing device, and
administer bilateral stimulation to the patient through the one or more visual and tactile stimulation components of the first and second handheld assemblies in accordance with the received control instructions.

2. The apparatus of claim 1, wherein the administration of bilateral stimulation to the patient comprises alternatingly activating the one or more visual and tactile stimulation components in the first handheld assembly and then the one or more visual and tactile stimulation components in the second handheld assembly at an oscillation speed set by the remote therapist.

3. The apparatus of claim 1, wherein the visual stimulation components of the first and second handheld assemblies comprise RGB light-emitting diodes (“LEDs”).

4. The apparatus of claim 3, wherein the control instructions comprise instructions to apply one or more of a color value and a brightness value to the visual stimulation components of the first and second handheld assemblies.

5. The apparatus of claim 1, wherein the tactile stimulation components of the first and second handheld assemblies comprise a motor with a counterweight integrated into the handheld assembly.

6. The apparatus of claim 5, wherein the control instructions comprise instructions to apply an intensity value to the tactile stimulation components of the first and second handheld assemblies.

7. The apparatus of claim 1, wherein the first handheld assembly and the second handheld assembly comprise egg-shaped or bicycle grip-like devices configured to be held in a left hand and a right hand, respectively, of the patient.

8. The apparatus of claim 7, wherein the microcontroller module is housed in a desktop assembly, and each of the first and second handheld assemblies are connected to the microcontroller module by a length of cable allowing the first and second handheld assemblies to be comfortably held by different sized patients in the left and right hands.

9. The apparatus of claim 1, wherein the computing device of the patient comprises a mobile phone, and wherein the microcontroller module is connected to the computing device by a Bluetooth connection.

10. A method of providing remote eye movement desensitization and reprocessing (“EMDR”) therapy to a patient, the method comprising:
connecting, by a computing device of the patient, to a cloud server hosting a therapist web application over one or more networks;
connecting, by the computing device and a remote EMDR device in possession of the patient, the computing device to the remote EMDR device;
receiving, by the therapist web application, control instructions for the remote EMDR device from a therapist;
sending, by the therapist web application, the control instructions to the computing device over the one or more networks;
forwarding, by the computing device, the control instructions to the remote EMDR device; and
administering, by the remote EMDR device, bilateral stimulation to the patient through one or more visual stimulation components and tactile stimulation components of the remote EMDR device based on the forwarded control instructions.

11. The method of claim 10, wherein the remote EMDR device comprises:
a desktop assembly housing a microcontroller module; and
a first handheld assembly and a second handheld assembly housing the one or more visual stimulation components and tactile stimulation components, each handheld assembly operably connected to the microcontroller module.

12. The method of claim 11, wherein the visual stimulation components of the first and second handheld assemblies comprise an RGB light-emitting diode (“LED”) integrated into each handheld assembly, the method further comprising:
receiving, by the therapist web application, control instructions for the remote EMDR device from the therapist to apply one or more of a color value and a brightness value to the visual stimulation components;
sending, by the therapist web application, the control instructions to the remote EMDR device via the computing device; and
applying, by the remote EMDR device, the one or more of the color value and the brightness value to the RGB LEDs of the first and second handheld assemblies.

13. The method of claim 11, wherein the tactile stimulation components of the first and second handheld assemblies comprise a vibration motor integrated into each handheld assembly, the method further comprising:
receiving, by the therapist web application, control instructions for the remote EMDR device from the therapist to apply an intensity value to the tactile stimulation components;
sending, by the therapist web application, control instructions to the remote EMDR device via the computing device; and
applying, by the remote EMDR device, the intensity value to the vibration motors of the first and second handheld assemblies.

14. The method of claim 11, wherein the first handheld assembly and the second handheld assembly comprise egg-shaped or bicycle grip-like devices configured to be held in a left hand and a right hand, respectively, of the patient.

15. The method of claim 11, wherein the computing device of the patient comprises a mobile phone, and wherein the remote EMDR device is connected to the computing device by a Bluetooth connection.

16. A system for providing remote eye movement desensitization and reprocessing (“EMDR”) therapy to a patient, the system comprising:
a remote EMDR device comprising a microcontroller module, a first handheld assembly, and a second handheld assembly, each handheld assembly operably connected to the microcontroller module and comprising one or more of a visual stimulation component and a tactile stimulation component;
a patient mobile device operably connected to the remote EMDR device; and
a cloud server operably connected to the patient mobile device over one or more networks and hosting a therapist web application configured to:
receive control instructions for the remote EMDR device from a therapist via a therapist console, and
send the control instructions to the patient mobile device over the one or more networks;
wherein the patient mobile device is configured to forward the control instructions received from the cloud server to the remote EMDR device, and the microcontroller module is configured to administer bilateral stimulation to the patient through the one or more visual and tactile stimulation component of the handheld assemblies based on the forwarded control instructions.

17. The system of claim 16, wherein the visual stimulation components of the first and second handheld assemblies comprise an RGB light-emitting diode (“LED”) integrated into each handheld assembly, the control instructions comprise an instruction to apply one or more of a color value and a brightness value to the visual stimulation components of the first and second handheld assemblies.

18. The system of claim 16, wherein the tactile stimulation components of the first and second handheld assemblies comprise a vibration motor integrated into each handheld assembly, the control instructions comprise an instruction to apply to apply an intensity value to the tactile stimulation components of the first and second handheld assemblies.

19. The system of claim 16, wherein the first handheld assembly and the second handheld assembly comprise egg-shaped or bicycle grip-like devices configured to be held in a left hand and a right hand, respectively, of the patient.

20. The system of claim 16, wherein the therapist console is configured to show a status of the remote EMDR device of the patient, the microcontroller module further configured to periodically send state telemetry data to the cloud server via the patient mobile device.
